(19) United States
(12) Patent Application Publication
(10) Pub. No.: US 2019/0343919 A1
(43) Pub. Date: Nov. 14, 2019

(54) METHODS FOR TREATING PERIPHERAL NERVE INJURY

(71) Applicant: UTI Limited Partnership, Calgary (CA)

(72) Inventors: Shalina S. Ousman, Calgary (CA); Erin-Mai F. Lim, Calgary (CA)

(73) Assignee: UTI Limited Partnership, Calgary, AB (CA)

(21) Appl. No.: 16/461,430

(22) PCT Filed: Nov. 16, 2017
(86) PCT No.: PCT/IB2017/057150

§ 371 Date: May 16, 2019

Related U.S. Application Data
(60) Provisional application No. 62/423,747, filed on Nov. 17, 2016.

(51) Int. Cl. A61K 38/17; A61P 25/00

(52) U.S. Cl. CPC A61K 38/1709; A61P 25/00

(57) ABSTRACT

AlphaB-crystallin (αBC) is a small heat shock protein that is constitutively expressed by peripheral nervous system (PNS) axons and Schwann cells. The present invention provides data on the role of alphaB-crystallin plays after peripheral nerve damage. Surprisingly and unexpectedly, the present inventors have also found that loss of αBC impaired remyelination which correlated with a reduced presence of myelinating Schwann cells and increased numbers of non-myelinating Schwann cells. The present inventors have also discovered that heat shock protein appears to regulate the crosstalk between Schwann cells and axons. Such dysregulations can lead to defects in conduction velocity and motor and sensory functions. Further, application of exogenous alphaB-crystallin or increased expression of alphaB-crystallin has a beneficial effect in peripheral nerve injury by augmenting remyelination and functional recovery in vivo. In general, it was discovered that αBC plays an important role in regulating Wallerian degeneration and remyelination following PNS injury.

### CROSS-REFERENCE TO RELATED APPLICATIONS

This application claims the priority benefit of U.S. Provisional Application No. 62/423,747, filed Nov. 17, 2016, which is incorporated herein by reference in its entirety.

### FIELD OF THE INVENTION

The present invention relates to a method for treating peripheral nerve injury. In particular, the present invention provides a method for remyelination of injured or damaged peripheral nerve cells. In some embodiments, the method involves using alphaB-crystallin.

### BACKGROUND OF THE INVENTION

Treatment of central nervous system (“CNS”) injury and peripheral nervous system (“PNS”) injury differs significantly. See, for example, Taveggia et al. in “Signals to promote myelin formation and repair,” Nature Reviews Neurology, 2010, 6, 276-287. For example, for CNS injury, potential therapies include enhancing the cell body response to injury, implantation of artificial conduits containing growth factors and cell adhesion molecules, application of stem cells, gene therapy and electrical nerve stimulation. In contrast, currently primary treatments for peripheral nerve injuries are surgical reconnection and/or rehabilitation therapy.

Peripheral nerve injury is damage of the peripheral nerves and commonly manifests as a form of hand, leg or facial dysfunction that is often associated with neuropathic pain that can be the more debilitating. Although, it is a common injury, the current treatment options rely on surgical anastomosis or nerve engraftment which often has non-optimal outcomes. Generally, in a closed or crush injury the recommendation is to wait 3 months to see if there is any improvement before attempting surgical repair. In open wounds (laceration) surgical repair, transfer or grafting is attempted immediately.

In a closed or crush peripheral nerve injury, optimal time for surgery is often missed as the decision for surgical treatments are made later after an initial diagnosis of the injury. Therefore, an effective treatment option is needed for peripheral nerve injury as delayed surgical repair can lead to only partial nerve regeneration.

### SUMMARY OF THE INVENTION

Regeneration of axons and full behavioral recovery of the damaged human peripheral nervous system is incomplete. The present invention is based on the discovery by the present inventors of importance of αBC for mediating remyelination of damaged peripheral nervous system (“PNS”) axons. In particular, administration of alphaB-crystallin, which is expressed by peripheral axons and Schwann cells, to damaged peripheral nerve cells resulted in a significant increase in remyelination. In contrast, absence of αBC resulted in thinner myelin sheaths and fewer myelinating Schwann cells, resulting in decreased nerve conduction and, sensory and motor behaviors.

One aspect of the invention provides a method for treating a subject suffering from a peripheral nerve damage or injury. The method comprises administering to a subject in need of such a treatment a therapeutically effective amount of a molecule that increases remyelination of injured or damaged peripheral nerve cells. In some embodiments, said molecule comprises alphaB-crystallin. Yet in other embodiments, the subject is treated with said molecule within 7 days, typically within 2 days, and often within 1 day of said peripheral nerve injury. Still in other embodiments, the subject is treated with said molecule for at least 7 days, typically for at least 14 days, and often for at least 28 days after said peripheral nerve injury. Such a method of treatment results in at least 60%, typically at least 70%, often at least 80%, more often at least 90%, still more often at least 95% improvement, and most often at least 100% improvement in remyelination of injured or damaged peripheral nerve cells compared to the absence of said treatment. Alternatively, such a method of treatment results in at least 80%, typically at least 90%, and often at least 100% improvement in sensory or motor activity or behavior in the subject 14 days after the initial peripheral nerve injury or damage.

In some embodiments, the peripheral nerve injury or damage comprises injury or damage to sacral plexus nerves (e.g., sciatic nerve, sural nerve, tibial nerve, common peroneal nerve, deep peroneal nerve, superficial peroneal nerve); lumbar plexus nerves (e.g., iliohypogastric nerve, ilioinguinal nerve, genitofemoral nerve, lateral cutaneous nerve, obturator nerve, femoral nerve); cranial nerves (e.g., olfactory nerve, optic nerve, oculomotor nerve, trochlear nerve, abducens nerve, trigeminal nerve, facial nerve, vestibulocochlear nerve, glossopharyngeal nerve, vagus nerve, hypoglossal nerve, accessory nerves); cervical plexus nerves (e.g., suboccipital nerve, greater occipital nerve, lesser occipital nerve, greater auricular nerve, lesser auricular nerve, phrenic nerve); brachial plexus nerves (e.g., musculocutaneous nerve, radial nerve, median nerve, axillary nerve, ulnar nerve); sympathetic nerves; and parasympathetic nerves and/or their distal branches.

Another aspect of the invention provides a method for treating a subject having injured or damaged peripheral nerve cell. Such a method comprises administering to a subject suffering from injured or damaged peripheral nerve cell a therapeutically effective amount of alphaB-crystallin. In some embodiments, the subject is treated with alphaB-crystallin within 7 days, typically within 2 days, and often within 1 day of suffering from injury or damage to peripheral nerve cell. Still in other embodiments, the subject is treated with alphaB-crystallin for at least 7 days, typically for at least 14 days, and often for at least 28 days after suffering from injury or damage to peripheral nerve cell.

Yet another aspect of the invention provides a method for treating a subject suffering from a peripheral nerve damage or injury, said method comprising treating said subject with a composition or a process to increase the expression level or availability of alphaB-crystallin. In one particular embodiment, said composition comprises a therapeutically effective amount of a molecule that increases remyelination of injured or damaged peripheral nerves. Yet in another embodiment, said process to increase the expression level or availability of alphaB-crystallin comprises heat treatment, oxidative stress, osmotic dysregulation, or blocking a pathway known to inhibit alphaB-crystallin expression. Still in another embodiment, said composition or process for increasing alphaB-crystallin expression or activity comprises heat, arsenite, phorbol 12-myristate 13-acetate, okadaic acid, H₂O₂, anisomycin, a high concentration of NaCl or sorbitol, or a combination thereof. Examples of stimuli that increase alphaB-crystallin expression is disclosed in J. Biol. Chem., 272 (1997), pp. 29934-29941, which is incorporated herein by reference in its entirety.

### BRIEF DESCRIPTION OF THE DRAWINGS

FIG. 1 shows results of expression of αBC in sciatic nerves before and after crush injury. Panel (A) is Western blot image showing expression levels of αBC in sciatic nerves from naïve 129SVE wild-type (“WT”) and αBC^{-/-} mice. Panel (B) is immunohistochemical staining for αBC, MBP, NF—H, GFAP, and DAPI in cross-sections of sciatic nerves from WT naïve mice. (Magnification: 40×; scale bar: 10 μm.). Panel (C) is Western blot image and ImageJ quantification of the levels of αBC and actin in sciatic nerves from WT naïve animals at 1, 3, 7, 14, 21, 28, and 56 days postcrush (representative of two experiments, with each bar consisting of four animals per time point). Data were analyzed using the independent t test comparison with the naïve time point and are shown as means±SEM. *P<0.05. Panel (D) is immunohistochemical staining for αBC in longitudinal sections of naïve and 7-d crushed WT nerves. (Scale bar: 10 μm.).

FIG. 2. shows sensory and motor behaviors in WT and αBC^{-/-} mice after sciatic nerve crush. DigiGait analysis of (A) swing duration, (B) stance duration, (C) braking duration, (D) propulsion duration, and (E) paw area in naïve and 28-d postcrushed WT (white bars) and αBC−/− (black bars) mice (representative of two experiments; n=3-5 mice per group). (F) RotaRod test performed on naïve (N; white and black circles) and 28-d injured (I; white and black triangles) WT (white symbols) and αBC^{-/-} (black symbols) mice (one experiment; n=9-10 animals per group). (G) SFI examination and SFI difference in WT (circles and white bar) and αBC^{-/-} (triangles and black bar) mice before (N) and 28 d after crush injury (I; representative of two experiments; n=9-10 mice per group). (H) Dynamic plantar test in naïve, sham, and 28-d injured WT (white bars) and αBC^{-/-} (black bars) mice (one experiment; n=9-10 animals per group). (I) von Frey Hair examination in sham (S) and 28-d injured (I) WT (circles) and αBC^{-/-} (triangles) animals (representative of two experiments; n=7-10 mice per group). (J) Hargreaves test in naïve (N) and injured WT and αBC^{-/-} mice at 28 and 56 d after sham (S) and injury (I) surgeries (representative of two experiments; n=7-10 mice per group). All data were analyzed using two-way repeated measures ANOVA and represent mean±SEM. *P<0.05.

FIG. 3. shows electrophysiological properties of motor axons in sciatic nerves of WT and αBC^{-/-} mice after crush injury. Panels (A and D) Latency, Panels (B and E) distance, and Panels (C and F) normalized latency in Panels (A-C) naïve (N) and Panels (D-F) 28-d postsurgery sham (S) and injured (I) WT (white bars) and αBC^{-/-} (black bars) mice after a single-point stimulation of the sciatic nerve (one experiment; n=5 per group). *P<0.05 (two-way ANOVA). Panel (G) is an example of the raw data for the normalized latency reflecting mean data represented in Panel F. The dotted line indicates the stimulus artifact, and the black arrows indicate the first poststimulus voltage deflections associated with the arrival of motor volley near the recording electrode. The latency was measured from the dashed line to the arrows. Trace is an average of 20 individual stimulus trials. Panel (H) shows MNCV in naïve, sham, and 28-d postdamaged WT (white bars) and αBC^{-/-} (black bars) animals (representative of two experiments; n=9-10 mice per arm). *P<0.05 (two-way repeated measures ANOVA). Panel (I) shows CMAP amplitude of sham (0) and 28-d injured WT and αBC mice. Panel (J) is representative traces of the raw data from which the CMAP data and MNCV were derived in sham (S) and 28-d injured (I) WT and αBC^{-/-} mice.

FIG. 4 shows remyelination in WT and αBC null mice after sciatic nerve injury. Bright field images of toluidine blue stained epon embedded sciatic nerve cross sections and g-ratio analyses in naïve (Panel A) and 28 d post-injured (Panel B) WT (white bars) and αBC^{-/-} (black bars) mice; representative of 2 experiments, n=3-5/group, magnification=100×, bar=10 μm. Data is displayed as g-ratio frequency distribution of WT and αBC^{-/-} mice and analysed using an independent t-test (*p<0.05).

FIG. 5 shows axonal characteristics of WT and αBC^{-/-} mice. Number of myelinated axons (Panel A) and axon cross sectional area (Panel B) in WT and αBCKO mice in 28 d post-crushed epon embedded sections stained with toluidine blue; representative of 2 experiments, n=3-5/group. (Panel C) Western blot image and Image J quantification of the levels of GAP-43 in naïve (N) and 28 d post-injured (I) sciatic nerves from WT (white bars) and αBC^{-/-} (black bars) mice; 1 experiment, n=3/group. (Panel D) Outgrowth of processes from DRGs isolated from WT and αBC null mice at 24 h in culture analysed for percentage of cells with neurites, mean number of processes/cell, mean outgrowth/cell and mean longest neurite/cell, magnification bar=200 μm. Outgrowth measures were compared using the independent t-test (unpaired, two-tailed) with statistical significance set at p<0.05.

FIG. 6 shows Schwann cell profile in injured WT and αBC^{-/-} animals. Representative images and quantification of the number of S100+(Panel A), GFAP+ (Panel B) and P0 (Panel C) profiles in the sciatic nerves of naïve and 28 d post-crushed WT (white bars) and αBC^{-/-} (black bars) animals; representative of 2 experiments, n=3-5 animals/group. (Panel D) Western blot image and Image J quantification of the levels of Krox-20 in naïve (N) and 28 d post-injured (I) sciatic nerves from WT (white bars) and αBC^{-/-} (black bars) mice; 1 experiment, n=3/group. All data represent mean±s.e.m., *p<0.05 independent t-test.

FIG. 7 shows expression of neuregulin, ErbB2 and AKT in injured sciatic nerves from WT and αBC^{-/-} mice. Western blot levels and image J analysis of neuregulin 1 Types I and III (Panel A), ErbB2 (Panel B) and AKT (Panel C) in WT and αBC^{-/-} animals before injury (N) and at various time points (3, 5, 7, 14, 28 d) after crush damage; 1 experiment, n=4/group. Displaying 2 animals per time point with each quantification time point consisting of 4 animals. All data represent mean±s.e.m., *p<0.05 independent t-test.

FIG. 8 shows therapeutic effect of recombinant human αBC in WT mice after sciatic nerve crush injury. (Panel A) Representative images of toluidine-blue stained epon embedded sections and g-ratio analysis from WT animals treated with PBS (white bar) or rhu-αBC (black bar) at 28 d post-crush; n=3-4/group, magnification=100×, bar=10 μm. (Panel B) Sciatic functional index examination and SFI difference in PBS (white circles and bar) and rhu-αBC-treated (black circles and bar) injured WT mice before (N-naïve) and 28 d after crush injury; representative of 2 experiments, n=9-10 mice/group. All data represent mean±s.e.m., *p<0.05 independent t-test. (Panel C) von Frey hair test in PBS (white circles and triangles) and rhu-αBC-treated (black circles and triangles) sham (S) and injured (I) WT animals tested before (N) and after crush (I); 1 experiment, n=10 mice/group. Data analysed using two way repeated measures ANOVA, *p<0.05 and represent mean±s.e.m.

FIG. 9 shows αBC expression in injured sciatic nerves. Cross sections of sciatic nerves at 7 d post-crush stained for αBC, F4/80, GFAP and Fizz1. Magnification=40×, bar=10 μm.

FIG. 10 shows a DigiGait system. Image of the DigiGait system (Panel A) and graphical depiction of various aspects of a mouse's gait (Panel B).

FIG. 11 shows assessment of Wallerian and Wallerian-like processes. Representative photos and quantification of the number of NF—H+ (Panel A), P0+(Panel B) and Iba-1+ (Panel C) profiles in naïve and 7 d crushed WT and αBC^{-/-} sciatic nerves. One experiment, n=4 animals/group, magnification=20×, bar=20 μm. Data represent mean±s.e.m., *p<0.05 independent t-test.

FIG. 12 shows evaluation of the crush injury paradigm. Images of cross (Panels A, B) and longitudinal (Panels C, D) sections from naïve (Panel A) and 7 d (Panels B-D) crushed WT nerves stained for NF—H. Magnification=20×, bar=50 μm (Panel A), 200 μm (B) and 100 μm (Panels C, D).

### DETAILED DESCRIPTION OF THE INVENTION

Some aspects of the invention are based on the discovery by the present inventors that administration of alphaB-crystallin (“αBC”) to a subject suffering from injury or damage to peripheral nerve promoted remyelination and functional recovery. In general any peripheral nerve injury or damage can be treated by the methods of the invention. As used herein, the term “treating injury or damage to peripheral nerve” refers to regaining at least a partial function of the peripheral nerves that have been injured or damaged. Exemplary peripheral nerve functions that can be regained by methods of the invention include, but not limited to, motor activity, sensory activity, etc. Generally, methods of the invention improves nerve function(s) by at least 50%, typically by at least 60%, often by at least 70%, more often by at least 80%, still more often by at least 90%, and most often by at least 95%. Such improvements can be measured, for example, by using a behavioral test as described in the Example section.

In some embodiments, methods of the invention can be used to treat injury or damage to sacral plexus nerves (e.g., sciatic nerve, sural nerve, tibial nerve, common peroneal nerve, deep peroneal nerve, superficial peroneal nerve); lumbar plexus nerves (e.g., iliohypogastric nerve, ilioinguinal nerve, genitofemoral nerve, lateral cutaneous nerve, obturator nerve, femoral nerve); cranial nerves (e.g., olfactory nerve, optic nerve, oculomotor nerve, trochlear nerve, abducens nerve, trigeminal nerve, facial nerve, vestibulocochlear nerve, glossopharyngeal nerve, vagus nerve, hypoglossal nerve, accessory nerves); cervical plexus nerves (e.g., suboccipital nerve, greater occipital nerve, lesser occipital nerve, greater auricular nerve, lesser auricular nerve, phrenic nerve); brachial plexus nerves (e.g., musculocutaneous nerve, radial nerve, median nerve, axillary nerve, ulnar nerve); sympathetic nerves; and parasympathetic nerves and/or their distal branches.

One particular aspect of the invention provides a method for treating a subject suffering from a peripheral nerve damage or injury by administering to a subject in need of such a treatment a therapeutically effective amount of a molecule that increases remyelination of injured or damaged peripheral nerve cells. In some embodiments, the molecule comprises alphaB-crystallin. In some embodiments, the subject is treated with said molecule within 7 days, typically within 2 days, and often within 1 day of said peripheral nerve injury. However, it should be appreciated that the time period for treating such an injury using methods of the invention is not limited to these time periods. In some cases, the treatment can be administered as soon as possible.

Still in other embodiments, the subject is treated with said molecule for at least 7 days, typically for at least 14 days, and often for at least 28 days. The treatment can be every few hours, every day, every other day, every few days, or can be intermittently administered. One skilled in the art having read the present disclosure can readily determine the treatment periods and/or frequency.

Methods of treatment results in at least 60%, typically at least 70%, often at least 80%, more often at least 90%, still more often at least 95% improvement, and most often substantially 100% improvement in remyelination of injured or damaged peripheral nerve cells compared to the absence of said treatment. Alternatively, such a method of treatment results in at least 50%, typically ate least 60%, often at least 70%, more often at least 80%, still more often at least 90%, and most often substantially 100% improvement in sensory or motor activity or behavior in the subject. Typically, such improvement can be observed within 7 days, typically within 14 days, often within 28 days and most often after two month after the initial treatment and/or peripheral nerve injury or damage.

Another aspect of the invention provides a method for treating a subject having injured or damaged peripheral nerve cell by administering to a subject suffering from injured or damaged peripheral nerve cell a therapeutically effective amount of alphaB-crystallin. In some embodiments, the subject is treated with alphaB-crystallin within 7 days, typically within 2 days, and often within 1 day of suffering from injury or damage to peripheral nerve cell. Still in other embodiments, the subject is treated with alphaB-crystallin for at least 7 days, typically for at least 14 days, and often for at least 28 days after suffering from injury or damage to peripheral nerve cell.

AlphaB-crystallin (HSPB5/CRYAB/αBC) is a small heat shock protein that enhances survival in response to stress by inhibiting protein aggregation, reducing levels of intracellular reactive oxygen species and inhibiting programmed cell death. AlphaB-crystallin has been found in malignant diseases such as gliomas, prostate, renal and breast carcinomas, and its expression has been associated with poor clinical outcomes in many cancers. In neurodegenerative disorders such as multiple sclerosis (MS), it has been reported to be up-regulated in oligodendrocytes in pre-active lesions, as well as astrocytes and microglia, and, to suppress the activation of innate and adaptive immune responses. Beneficial effects of alphaB-crystallin in a mouse model of MS have also been reported; however, the therapeutic potential of this protein in the repair of peripheral nerve injury has not been well-described.

αBC is expressed constitutively by the peripheral nervous system (PNS) axons and Schwann cells. The inventors have found that loss of the crystallin impaired conduction velocity and, motor and sensory functions. Without being bound by any theory, it is believed that this effect is due to deficits in remyelination.

The robust regenerative capacity of the damaged peripheral nervous system (PNS) is partly determined by cellular and molecular events that occur in the nerve segment distal to the injury site. For instance, during Wallerian degeneration, influx of calcium into the damaged nerve within 12-24 h of PNS injury, activates proteases that result in cytoskeletal breakdown and subsequent disintegration of the axon membrane. This is then followed by breakdown of the myelin sheath within two days. Schwann cells, the glial cells that characterize the PNS, subsequently undergo a number of reactive physiological changes that benefit the damaged axon. Within 48 h of peripheral nerve damage, myelinating Schwann cells decrease their expression of myelin proteins such as myelin basic protein (MBP), peripheral myelin protein 22 (PMP22) and protein 0 (P0) and along with their non-myelinating counterparts, revert to a non-myelinating phenotype. At approximately 3-4 days post-injury, the de-differentiated Schwann cells proliferate and align within the basal lamina to form bands of Büngner that provide a structural and trophic supportive substrate for regenerating axons. These Schwann cells secrete neurotrophic factors that provide trophic sustenance to damaged neurons until they reestablish contact with their targets, produce extracellular matrix molecules that encourage and guide outgrowing axons, while secretion of chemokines are thought to mediate the infiltration of blood-derived macrophages which, along with Schwann cells, phagocytose myelin debris and its associated axon growth inhibitors. Finally, based on the level of neuregulin I Types I and III on Schwann cells and axons, respectively, and, their binding to their cognate receptors ErbB2/ErbB3 on Schwann cells, these glia will revert to a myelinating or ensheathing phenotype upon contact with regrowing axons. These morphological and physiological changes in Schwann cells create an environment that encourages long distance axon growth.

In humans however, regrowth of damaged peripheral nerves is often incomplete and this can result in partial or complete loss of motor, sensory and autonomic functions, neuropathic pain, or inappropriate sensations. This insubstantial regrowth of damaged peripheral axons in humans is attributed to a variety of factors: 1) the slow rate of axon regrowth (˜1 mm/day), 2) the often far distances of the injury site from the target, 3) the severity of the injury (transection versus crush where there is a complete loss of connective tissue in the former), 4) lack of selective axon-target reconnection, 5) nerve gap distance where gaps longer than 4 cm preclude recovery almost completely, 6) an inability of denervated muscles to accept reinnervation, 7) extensive associated injuries such as vascular disruptions, 8) older age of the individual and, 9) deterioration of the growth supportive abilities of Schwann cells. Methods of the invention are results of at least in part on experiments conducted by the present inventors to understand what regulates the beneficial processes of Schwann cells so as to improve regeneration and functional recovery in humans after damage to peripheral axons.

Schwann cells, as well as cell bodies and axons of peripheral neurons, are known to constitutively express a small heat shock protein called alphaB-crystallin (αBC) but its function in the uninjured and damaged PNS is unknown. αBC (also called CRYAB or HSPB5) is a 22 kDa protein that possesses a number of beneficial and protective properties including chaperoning, pro-survival, immunosuppression and anti-neurotoxic abilities. With respect to the PNS, others have reported that αBC was expressed late during PNS development and, that the heat shock protein was highly expressed in mature peripheral nerves with equal levels in both myelinating and non-myelinating Schwann cells. Further, expression of αBC was upregulated during PNS myelination and downregulated in cut rat sciatic nerves.

αBC has been shown to be therapeutic after spinal cord contusion injury in mice whereby treatment with recombinant human CRYAB post-damage resulted in reduced secondary tissue damage and greater locomotor recovery. However, its potential role in PNS injury has not been studied. It is well recognized by one skilled in the art that treatment methods and/or mechanism for CNS injuries are significantly different from PNS injuries. See, for example, Taveggia et al., in Nature Reviews Neurology, 2010, 6, 276-287; and Lutz et al. in “Contrasting the glial response to axon injury in the central and peripheral nervous systems,” Developmental Cell, 2014, 28(1), 7-17. Recently, studies have shown that αBC was therapeutic after spinal cord contusion injury in mice whereby treatment with recombinant human CRYAB post-damage resulted in reduced secondary tissue damage and greater locomotor recovery. In light of its expression in the PNS, together with its numerous beneficial and protective functions, the present inventors have investigated whether αBC influenced the injury-related events that occur after PNS damage.

Surprisingly the present inventors have discovered that αBC is important for remyelination of regenerated peripheral axons by regulating the conversion of de-differentiated Schwann cells back to a myelinating phenotype. The present inventors have also found that this heat shock protein contributes to the early communication between Schwann cells and damaged axons to signal that an injury has occurred. In addition, exogenous application of αBC provided therapeutic capabilities by promoting remyelination and functional recovery after PNS injury.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

I. Mice:

αBC-null (αBC^{-/-}) mice generated from embryonic stem (“ES”) cells with a 12954/SvJae background and maintained in 12956/SvEvTac X 12954/SvJae background. αBC^{-/-} mice are viable and fertile, with no obvious prenatal defects and normal lens transparency. Older mice show postural defects and progressive myopathy that are apparent at approximately ˜40 weeks of age. These animals were studied between 8-12 weeks thus removing the possible effects of myopathy on behavioral evaluation. Further, analyses on age-matched uninjured 129S6/SvEvTac wildtype (WT) and αBC^{-/-} were performed before injury to confirm equivalent baseline properties. Colonies of WT and αBC^{-/-} mice were bred and maintained in a facility that maintained a 12 h light/12 h dark cycle. Mice were housed at a maximum of 5 animals per cage.

II. Surgery:

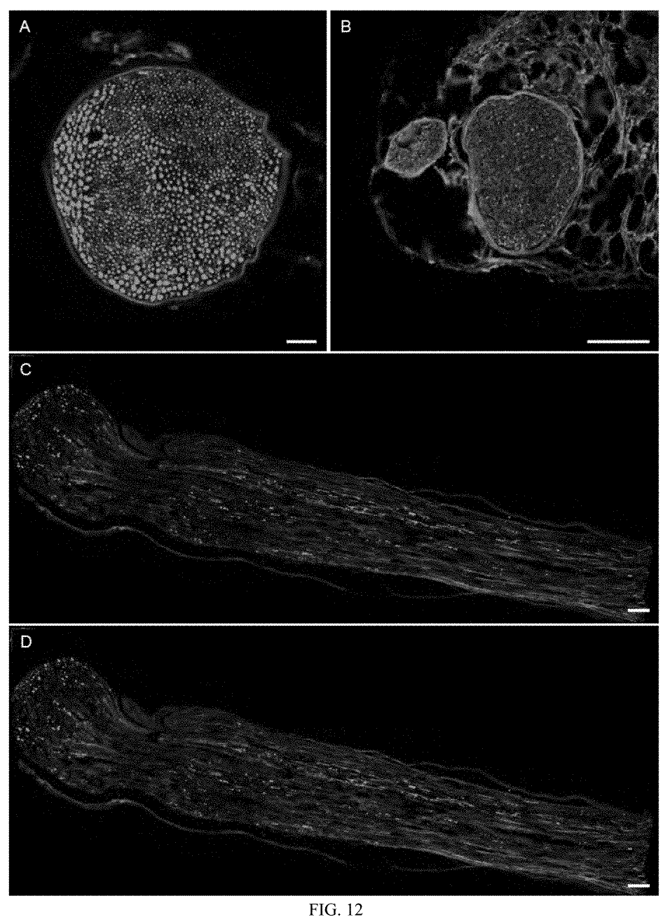

Eight to twelve week old female WT and αBC^{-/-} mice were anesthetized with a 3:1 ketamine:xylazine (200 mg/kg:10 mg/kg) mixture by intraperitoneal injection. An incision was made through the skin below the hip and the muscle was blunt dissected using fine surgical scissors and forceps to expose the right sciatic nerve at mid-thigh level. The nerve was crushed 0.5 cm above the region where it splits into the sural, common peroneal and tibial branches. For crushing, the sciatic nerve was first compressed with a straight tip serrated 5.0 fine forceps for 30 s. To ensure that the majority of axons were damaged, the forceps were then rotated 90° and the same area crushed again for an additional 30 s until a translucent region was evident. Evidence that the majority of axons sustained injury is shown in FIG. 12, where high expression of NF—H that is typically seen in intact nerves was markedly reduced in damaged fibers particularly at and around the crush site. Also, punctate, irregular NF staining distal to the crush site was observed that is likely axonal debris (FIG. 12 panel D). Animals were allowed to recover on a heated pad and sacrificed at 1, 3, 5, 7, 14, 21, 28, or 56 d post-injury. Naïve represents mice that have not undergone any surgical manipulation, whereas sham refers to undamaged nerves on the contralateral side of unilaterally crushed mice, where only the skin and muscles overlying the sciatic notch area were incised.

III. Western Blotting:

In total, 30-50 μg total protein was subjected to 5-15% sodium dodecyl sulfate polyacrylamide gel electrophoresis (SDS/PAGE), transferred to polyvinylidene fluoride (PVDF) membranes, and blocked with 5% (wt/vol) nonfat dried milk in Tris-hydrochloride (HCl)-buffered saline containing 0.05% Tween-20. Membranes were immunoblotted overnight at 4° C. with the following primary antibodies: Ms anti-GAP-43 (MAB347; 1:400; Millipore), Rb anti-αBC (ABN185; 1:1,000; EMD Millipore), Rb anti-actin (A2006; 1:1,000; Sigma-Aldrich), Rb neuregulin 1 Type I (sc-348; 1:500; Santa Cruz), Ms neuregulin 1 Type III (MABN42; 1:1,000; Millipore), Sh ErbB2 (AF5176; 1:1,000; R&D), Ms p-ErbB2 (04-294; 1:1,000; Millipore), Rb AKT (9272; 1:1,000; Cell Signaling), Rb p-AKT (4060; 1:1,000; Cell Signaling), Rb p38 (9212; 1:1,000; Cell Signaling), Ms p-p38 (9216; 1:2,000; Cell Signaling), Rb ERK (9102; 1:1,000; Cell Signaling), Rb p-ERK (9101; 1:1,000; Cell Signaling), Rb JNK (9252; 1:1,000; Cell Signaling), and Rb p-JNK (9251; 1:1,000; Cell Signaling). Bound primary antibodies were visualized with horseradish peroxide (HRP)-conjugated anti-rabbit IgG, anti-mouse IgG (1:5,000; GE Healthcare), or anti-sheep IgG (1:1,000; R&D) followed by chemiluminescence detection using an electrochemiluminescence (ECL) Kit (Pierce).

IV. Western Blot Densitometric Quantification:

Western blot bands were quantified using ImageJ software. Briefly, arbitrary pixel units were obtained for the optical density (OD) of an area around each band and a ratio of OD:area was calculated. The OD:area values for a protein of interest were then normalized to the corresponding actin OD:area numbers. In order to compare across time points, values for all time points were normalized to the naïve protein and actin levels.

V. Immunohistochemistry:

Sciatic nerves were fixed in Zamboni's fixative, cryoprotected in 30% (wt/vol) sucrose solution, and frozen; 10-μm-thick sections were blocked with 0.1% Triton X-100 and 10% (vol/vol) normal goat serum followed by overnight incubation at 4° C. with the following primary antibodies: Rb anti-αBC (ABN185; 1:200; Chemicon), Ms anti-total-NF—H (2836; 1:400; Cell Signaling), Ms anti-MBP (SMI94; 1:500; Covance), Rb anti-GFAP (Z0334; 1:500; DAKO), Rb anti-myelin protein P0 (ABN363; 1:200; Millipore), Ms anti-Asma (A5228; 1:200; Sigma-Aldrich), Rt anti-F4/80 (MCA497EL; 1:200; AbD Serotec), Ms anti-non-p-NF—H (SMI-32R; 1:200; Covance), Rb anti-Iba-1 (019-19741; 1:200; Wako Chemicals), Ms anti-S100 (S2532; 1:500; Sigma-Aldrich), and DAPI (D3571; 1:2,000; Invitrogen). Bound antibody was detected using the following Invitrogen secondary antibodies at 1:200: anti-mouse 594 (A11005), anti-mouse 488 (A11007), anti-rabbit 594 (A11012), and anti-rabbit 488 (A11008).

VI. Immunohistochemistry Quantification:

Whole cross-sectional areas of the sciatic nerve were obtained at 20× magnification with an Olympus Slide Scanner microscope. The numbers of GFAP+, P0+, and S100β+ were quantified in an area of 250 um² in the middle of each sciatic nerve cross-section using the CellSens digital imaging software (Olympus). The quantifier was masked to the genotypes during counting.

VII. Remyelination and g-Ratio Analysis:

Sciatic nerves were removed and immersed in 2.5% gluteraldehyde in 0.1 M cacodylate buffer, pH 7.4, at 4° C. overnight. Nerves were then post-fixed in 2% osmium tetroxide in 0.1 M cacodylate buffer, pH 7.4, for 2.5 hours at room temperature and then embedded in Epon after alcohol dehydration. Semithin sections were stained with toluidine blue. Sections were examined on an Olympus BX51 upright microscope at 100× magnification. G-ratios of the images were analyzed using the ImageTrak software created by Dr. P. K. Stys (ucalgary.ca/styslab/imagetrak). The g-ratios of seven areas of each sciatic nerve were analyzed. Using the Autotrace Polygon Tool to identify myelinated axons, measurements of the inner area (axon), outer area (total fiber), and g-ratio were computed. The g-ratio is obtained by dividing the diameter of an axon by the diameter of the axon plus myelin sheath. Thus, a low g-ratio signifies thickly myelinated axons while fibers with thin myelin sheaths have a large g-ratio. The analyser was masked to the genotypes during quantification.

VIII. DRG Neuron Isolation, Staining and Quantification:

(i) Isolation.

L4-L6 DRGs were digested in a 0.1% collagenase/L15 solution for 60 min at 37° C. Debris was removed by density gradient centrifugation (100×g for 6 min), and cells were resuspended in Dulbecco's modified Eagle medium (DMEM)/F12. Neurons were plated in triplicate on a glass substrate coated with poly-L-lysine (0.01%) and mouse laminin (10 μg/mL) and allowed to adhere for 10 min followed by the addition of culture medium. Cells were then incubated at 37° C. in 5% CO₂ for 18 h.

(ii) Staining.

Cultured neurons were fixed in 37° C. 4% (wt/vol) paraformaldehyde (PFA)/1×PIPES Hepes EGTA MgSO₄ (PHEM) buffer, blocked with 5% (vol/vol) goat serum/1×PBS (20 min), and labeled with a primary antibody mixture of Ms anti-NF200 (N0142; 1:800; Sigma-Aldrich) and Ms anti-βIII-tubulin (801201; 1:1,000; BioLegend) to visualize neurites. Bound antibody was detected using the secondary antibody Alexa 488 (A11001; 1:200) and then, mounted on a glass slide using Vectashield mounting medium with DAPI nuclear stain. Neurite outgrowth was quantified using the Neurite Outgrowth function of theMetaXpress (Molecular Devices) software.

IX. Electrophysiological Assessment:

Normalized distal motor latencies and motor nerve conduction velocity were performed in naïve and 28 d post-injury animals. For normalized distal motor latencies, the sciatic nerve was stimulated just above the sciatic notch using bipolar hook electrodes and the electromyogram (EMG) activity was recorded (100×, 100 Hz-1 kHz) using bipolar recording electrodes inserted into the first dorsal interosseous muscle of the corresponding hind limb. The latency to record a compound muscle action potential (CMAP) from the dorsal interosseus muscle is called the distal latency. The conduction delay was measured from the onset of the stimulus artifact to the upward deflection of the CMAP. Normalized distal motor latencies were calculated by dividing the latencies by the distance from the stimulation to the recording site. These latencies depend on distal motor axon conduction velocity, neuromuscular transmission delay and muscle activation. The experimenter was masked to the genotypes during recording.

To calculate motor nerve conduction velocity, both the sciatic notch and knee were stimulated and the CMAPs were recorded from the tibial-innervated dorsal interossei foot muscles using subdermal needle electrodes. Conduction velocity of the nerve was calculated by dividing the difference in distance between the knee and the sciatic notch knee stimulating site divided by the difference in the latencies of the respective CMAPs. The body temperature of animals was kept constant at 37±0.5° C. throughout the experiment using a heating lamp.

X. Behavioral Tests:

All behavioral testing was performed in the light cycle.

DigiGait:

The DigiGait Imaging System (Mouse Specifics, Inc.) was used to assess gait dynamics before crush injury and 28 d post-damage (26). WT and αBC^{-/-} mice were placed on a motorized treadmill within a plexiglass compartment. Digital video images were acquired at a rate of 80 frames per second by a camera mounted underneath the treadmill to visualize paw contacts on the treadmill belt. The treadmill was set at a fixed speed of 15 cm/sec, which was determined as the baseline for both WT and αBC^{-/-} mice. The DigiGait software calculates values for multiple gait parameters including swing duration, braking duration, propulsion duration and paw area.

Sensory Function:

Prior to testing the behavioral responses to thermal or mechanical stimuli, mice were habituated to the test environment for 30 minutes. To assess thermal sensitivity (Hargreaves Test), hind paw withdrawal latencies to a radiant heat lamp were determined as the average of three measurements per paw over a 30-minute test period. Mechanical sensitivity was assessed using von Frey hairs ranging from 0.027-3.63 grams. The series of von Frey hairs was applied from below the platform to the plantar surface of the hind paw in ascending order beginning with the lowest weight hair. The hair was applied until buckling occurred and then maintained for 2 seconds. A trial consisted of application of the von Frey hair to the hind paw 5 times at 5-second intervals. If withdrawal did not occur during five applications of a particular hair, the next larger hair in the series was applied in a similar manner. The withdrawal threshold was determined as withdrawal from a particular hair 4 or 5 times out of the 5 applications. Three trials were run for each of the left (sham) and right (injured) hind paws.

Motor Behavior. (i) Rotarod:

Prior to testing, mice were left to habituate in the testing room for 30 minutes. Mice were trained on the rotarod for three days and three trials per day for a maximum time of three minutes and five-minute inter-trial intervals. Mice were gently placed onto the RotaRod by gently swinging the mouse by the tail onto the rotating rod. Once the mice were on the rotating rod, the lever was raised to start the trial. On the first few trials the rod was set at a low speed of 4 rpm and then eventually increased to 12 rpm (training speed). On the day of testing, the RotaRod was set to accelerating mode, which is a speed of 4-40 rpm over 5 minutes. The mice were placed on the rod and the testing started once they were in place. Each mouse was given one trial for a maximum of 5 minutes. The latency to fall was recorded, and the speed at which the mice fell for each trial.

(ii) Dynamic Plantar:

Prior to testing, mice were left to settle in the testing room for 30 minutes. Before the actual test, mice were given 3 habituation sessions—mice were placed in an overturned clear cup on a mesh grid for 15 minutes. On the day of testing, the dynamic plantar aesthesiometer was calibrated using a zero, five and fifty-gram weight. Mice were placed in cups for 15 minutes on the grid until they were settled and quiet. Each mouse was given three trials on each hind paw—alternate hind paws for each trail and five-minute wait between trials. Using the mirror, the probe was directed to the center plantar surface of the paw. A response of the latency to respond in seconds and the force in grams is recorded automatically by the dynamic plantar aesthesiometer.

(iii) Walking Track Analysis/Sciatic Functional Index:

Gait was analyzed by a 4×6×50 cm corridor in which a 50 cm long piece of white paper was placed on its base. Non-toxic red food coloring was painted onto the hind paws of each mouse. After two practice trials, the mice walked into a covered box at the far end without hesitation. Two test trials were obtained for each mouse. Using the known method footprints were analyzed by the following four measurements: distance to the opposite foot (TOF), footprint length (PL), maximal toe spreading between first and fifth toes (TS), and paw spreading between the center of the second and fourth toes (IT). Measurements were taken for both the normal leg (N) and the experimental leg (E). The formula for calculating the SFI is:

$$SFI = \vert \frac{( ETOF - NTOF )}{NTOF} + \frac{( NPL - EPL )}{EPL} + \frac{( ETS - NTS )}{NTS} + \frac{( EIT - NIT )}{NIT} \vert \times \frac{220}{4}$$

An index of zero reflects normal function and an index of ±100 theoretically represents complete loss of function.

XI. Therapeutic Application of αBC:

Uninjured 8 week old female WT mice underwent walking track analysis to establish gait baselines for each mouse. Sciatic nerve crush injuries were then performed and animals injected with either 10 μg of recombinant human αBC (USBiologicals, Salem, Mass., USA, Cat # C7944-53) diluted in 100 μL saline or 100 μL of saline as control every other day for 4 weeks for a total of 14 injections. At the end of the treatment period, animals underwent walking track analysis again before their nerves were processed for epon embedding. Semithin sections were stained with toluidine blue and g-ratio analyses performed.

XII. Statistical Analysis:

Data are presented as means±s.e.m. A two-tailed, independent Student's t-test (n=2 groups) was used to detect between-group differences. ANOVA was employed for n>2 groups while a two way repeated measures ANOVA was implemented for repeated measures. p<0.05 was considered.

Results

αBC is Expressed by Schwann Cells and Axons and, its Level and Expression are Decreased in Sciatic Nerves Following Crush Injury:

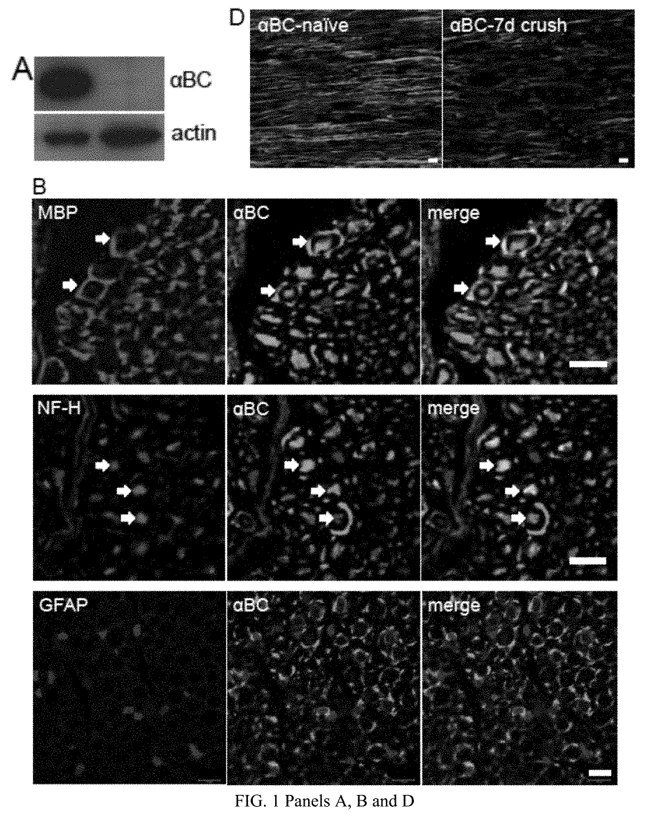

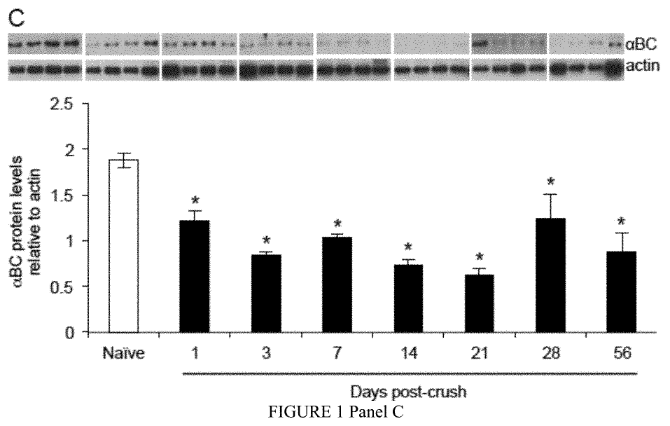

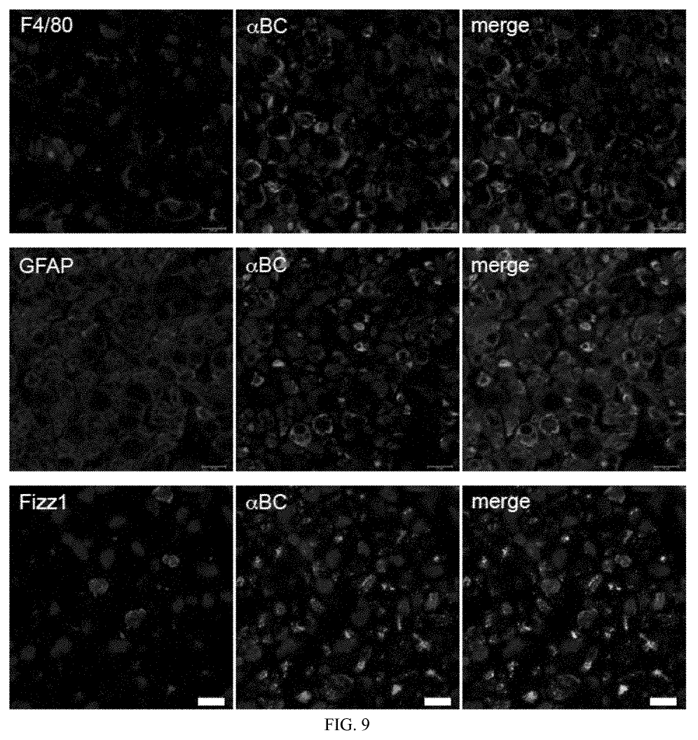

It has been shown that αBC is expressed constitutively in peripheral axons and Schwann cells from rat. The present inventor first checked to see if the heat shock protein was expressed in peripheral nerves from mice. High levels of αBC were evident in sciatic nerves from naïve 129S6 WT mice (FIG. 1 panel A) with co-localization to MBP positive Schwann cells and neurofilament-H stained axons (FIG. 1 panel B). No localization of the heat shock protein was seen in GFAP+ non-myelinating Schwann cells, F4/80+ macrophages or Fizz1+ fibroblasts (FIG. 1 panel A, FIG. 9). As expected, the crystallin was absent in nerves from αBC null animals (FIG. 1 Panel A). Whether the levels of the heat shock protein were altered after sciatic nerve crush was also evaluated. Within 24 h of damage in WT animals, there was a significant decrease in the amount of αBC in the nerve segment distal to the crush site (FIG. 1 panel C). This reduction was sustained until approximately 28 d post-injury after which the levels started to rebound relative to the lowest expression seen at day 21 after crush (FIG. 1 panel C). These data were confirmed at the histological level where a reduction in αBC immunostaining was evident at 7 d after crush injury as compared to intact nerves (FIG. 1 panels B, D; FIG. 9).

Sensory and Motor Behaviours are Impaired in αBC^{-/-} Mice after Sciatic Nerve Crush:

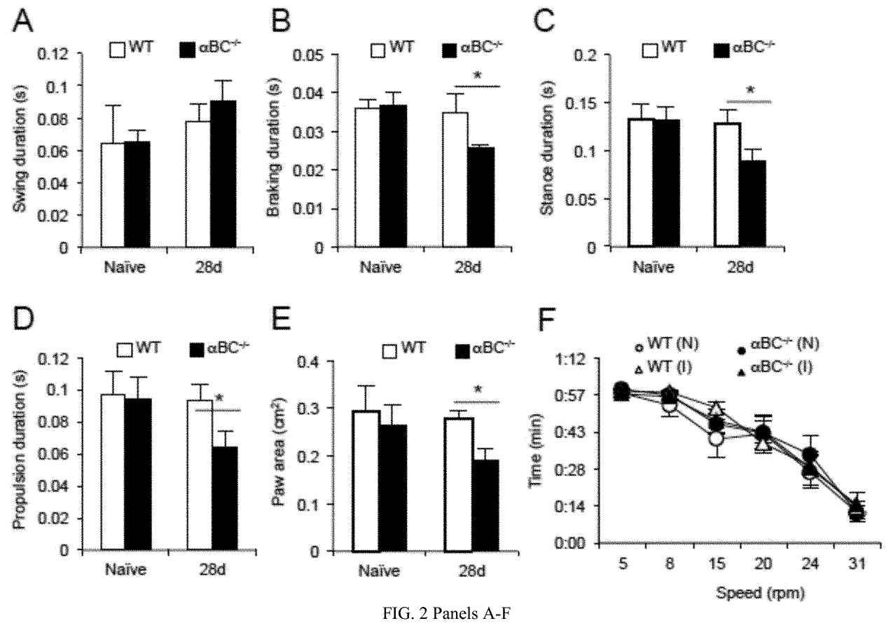

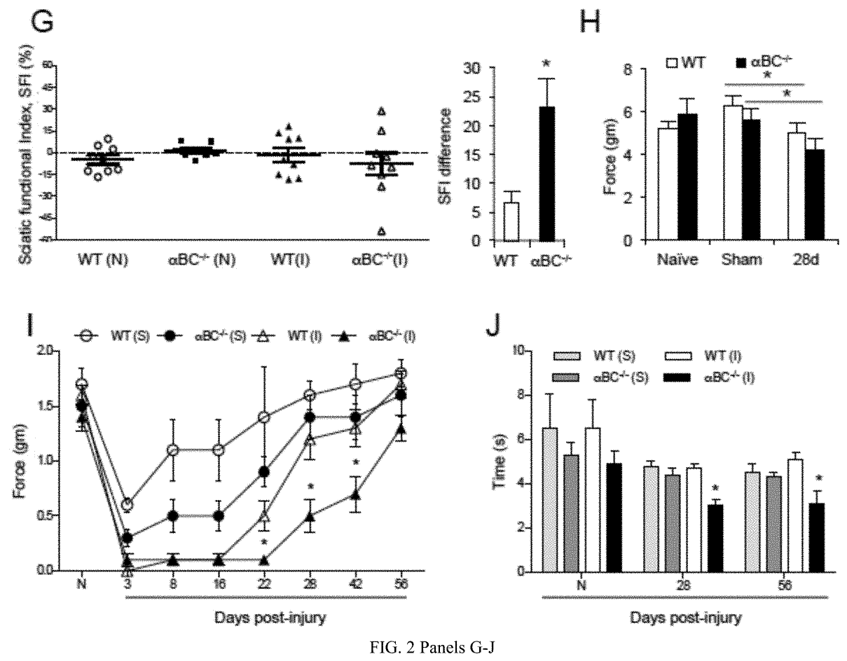

To assess whether removal of αBC impacted functional recovery, motor and sensory behaviors associated with sciatic nerve regeneration was evaluated at 28 days post-crush, a time point when regeneration is robust in WT mice and, when the levels of αBC were rebounding in the crushed sciatic nerves of WT mice (FIG. 1 panel C). To obtain an overall impression of effects on gait dynamics, the DigiGait Imaging System was used to assess parameters associated with regeneration such as swing, braking, propulsion, stance and paw area (FIG. 9, Table 1). It was first determined that the speed of the treadmill that allowed for proper gait dynamics in 129S6 strain of mice was 15 cm/sec. Then movement properties were compared in uninjured two month old WT and αBC null animals (age at which crush injury was performed) to assess whether there were any pre-existing differences in walking dynamics. No significant difference in swing duration (FIG. 2 panel A), stance duration (FIG. 2 panel B), braking duration (FIG. 2 panel C), propulsion duration (FIG. 2 panel D) or paw area (FIG. 2 panel E) was seen between the two uninjured, naïve groups (Table 2). The same mice were then re-tested 28 days after a crush injury. In WT animals, the gait parameters had recovered and were comparable to those of the naïve WT group. In injured αBC knockout mice however, braking duration, propulsion duration, paw area and stance duration were significantly lower relative to their WT counterparts (FIG. 2 panels B-E, Table 1) while swing duration had recovered (FIG. 2 panel A, Table 2). These results indicate that overall functional recovery was impaired in the injured αBC^{-/-} animals.

TABLE 1
Description of DigiGait System parameters
relevant to nerve regeneration

| Index | Meaning | Units | Definition |
|---|---|---|---|
| Swing | Swing | ms | Time duration of the |
| Duration | duration | | swing phase (no paw |
| | | | contact with belt) |
| % Swing | % of stride | % | % of the total stride |
| | in swing | | duration that the |
| | | | paw is in the air |
| Braking | Braking | ms | Time duration of the |
| Duration | duration | | braking phase (initial |
| | | | paw contact to maximum |
| | | | paw contact, commencing |
| | | | after the swing phase) |
| % Braking | % of stride | % | % of the total stride |
| | in braking | | duration that the paw |
| | | | is in the braking phase |
| Propulsion | Propulsion | ms | Time duration of the |
| Duration | duration | | propulsion phase |
| | | | (maximum paw contact |
| | | | to just before the |
| | | | swing phase) |
| % Propulsion | % of stride in | % | % of the total stride |
| | propulsion | | duration that the paw |
| | | | is in the propulsion phase |
| Stance | Stance | ms | Time duration of the |
| Duration | duration | | stance duration (paw |
| | | | contact with belt). |
| | | | Stance duration is equal |
| | | | to the sum of braking |
| | | | duration and propulsion |
| | | | duration |
| Stance | Width between both | cm² | The perpendicular distance |
| Width | forelimbs or both | | between the centroids of |
| | hindlimbs | | either set of axial paws |
| | | | during peak stance |
| Stance | Coefficient | % | The standard deviation |
| Width CV | of variation | | of the stance width for |
| | of stance width | | the set of strides recorded |
| | | | (reflecting the dispersion |
| | | | about the average value) |
| Paw Area | Paw area | cm² | The area seen by the |
| | | | camera, and reported |
| | | | at the time corresponding |
| | | | to peak stance (e.g., |
| | | | maximal paw area) |

TABLE 2
DigiGait System regeneration relevant motor and sensory behavioral parameters
in WT and αBC^{-/-} mice following sciatic nerve crush injury.

| | Naïve WT | Naïve αBC^{-/-} | Injured WT | Injured αBC^{-/-} |
|---|---|---|---|---|
| Gait Index | (n = 5) | (n = 5) | (n = 5) | (n = 5) |
| Hindlimb Swing (%) | 30.1 +/− 4.5 | 33.6 +/− 0.7 | 37.6 +/− 3.3 | 50.0 +/− 2.1**/*** |
| Hindlimb Braking (%) | 20.5 +/− 3.5 | 19.5 +/− 1.5 | 17.1 +/− 1.1 | 14.9 +/− 1.8 |
| Hindlimb Propulsion (%) | 49.4 +/− 3.6 | 47.0 +/− 1.9 | 45.3 +/− 2.9 | 35.1 +/− 2.1**/*** |
| Hindlimb Stance Width CV (%) | 30.6 +/− 6.0 | 35.2 +/− 3.2 | 36.5 +/− 8.1 | 55.9 +/− 3.6**/*** |

Data are means +/− s.e.m.;
*p < 0.05. compared to Naïve WT
Data are means +/− s.e.m.;
**p < 0.05, compared to Naïve αBC^{-/-}
Data are means +/− s.e.m.;
***p < 0.05, compared to Injured WT

Reduction in paw area, braking duration and propulsion duration is indicative of sensory and motor impairment. Therefore, the DigiGait findings was validated using classical motor (rotarod, walking track) and sensory (Hargreaves, Dynamic Plantar, von Frey Hair) behavioral tests. In the rotarod examination which measures motor coordination, no significant difference was observed between naïve and 28 d post-injured WT and αBC null animals (FIG. 2 panel F). In the walking track test which analyzes walking patterns to assess sensory-motor functionality during locomotion, WT animals showed some abnormality in walking at 28 d post-crush as evidenced by the sciatic functional index (SFI) scores that showed a range difference of 6.50±2.12 (FIG. 2 panel G). However, an even greater statistically significant exaggeration in walking impairment was seen in the αBC^{-/-} mice which displayed an SFI difference of 23.10±4.90 (FIG. 2 panel G). Both uninjured, naïve WT and αBC^{-/-} mice displayed SFI values close to zero which indicates that there were no developmental abnormality in gait in the null animals (FIG. 2 panel G).

Effects on sensory behavior were then assessed with the Dynamic Plantar, Hargreaves and von Frey Hair tests. For the dynamic plantar exam, which is a measure of mechanical sensitivity, while differences were observed between the sham and injured groups for both genotypes, no difference was evident between WT and null cohorts at 28 days after injury (FIG. 2 panel H). The von Frey Hair test which is another but more sensitive examination for mechanical sensitivity was also used for evaluation. Here, while both WT and αBC^{-/-} mice displayed increased sensitivity at the earlier time points post-injury (3, 8, 16 days), WT mice began to recover by day 22 to pre-injury values and fully recovered by day 28. However, αBC^{-/-} mice only started to recover by day 28, and remained sensitive to lower forces until 56 days post-crush (FIG. 2 panel I). In the Hargreaves test which measures for sensitivity to radiant heat, no difference in paw withdrawal was observed between uninjured WT and αBC^{-/-} animals. At 28 d and 56 d post-crush however, the injured null animals displayed increased sensitivity compared to their WT cohorts as seen by the reduced time in limb withdrawal (FIG. 2 panel J). The functional examinations revealed that the αBC^{-/-} animals displayed impairment in motor and sensory behaviors compared to their WT cohorts after sciatic nerve crush injury.

Conduction Velocity is Reduced in Sciatic Nerves from αBC Mice after Sciatic Nerve Crush Injury:

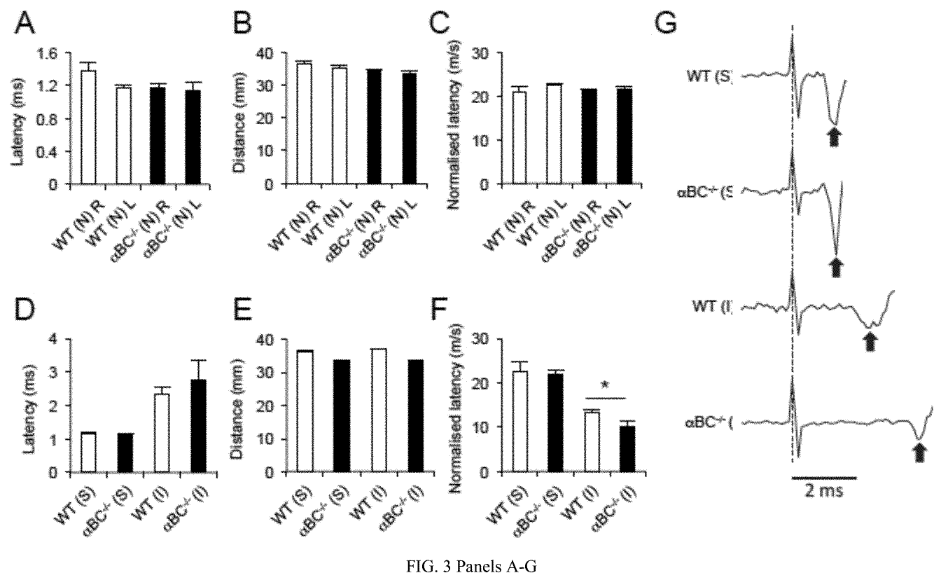

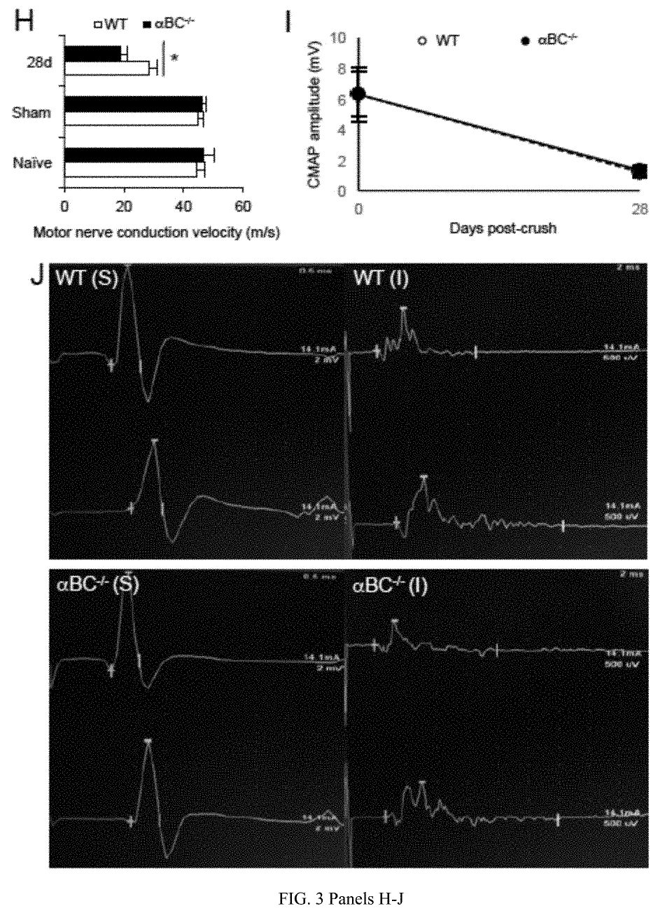

Whether the behavioral deficits seen in the injured αBC null mice were related to impairment in nerve conduction was also evaluated. The normalized distal motor latency of the sciatic—dorsal interosseus motor system in naïve and injured WT and αBC^{-/-} animals were assessed. No significant difference in normalized latency was seen between naïve, uninjured WT and null animals at a fixed distance (FIG. 3 panels A-C). Twenty-eight days after crush injury, αBC^{-/-} mice displayed a greater latency (FIG. 3 panel D) as compared to the WT cohort over a similar distance (FIG. 3 panel E). When the normalized latency was calculated, the null cohort displayed a reduction compared to injured WT controls (FIG. 3 panels F, G) indicating that recovery was less robust in injured αBC^{-/-} animals.

To determine whether the impairment in normalized latency in αBC^{-/-} mice was specifically related to axonal electrophysiological properties, independent of neuromuscular junction transmission, the motor nerve conduction velocity (MNCV) was measured. It was found that null mice displayed lower MNCV at 28 days post-crush as compared to the injured WT cohort (FIG. 3 panel G). This effect was specific to the injury process and not due to an underlying genetically-mediated influence because no difference in MNCV was seen in naïve or sham WT and αBC^{-/-} animals (FIG. 3 panel G). Further, it appeared that the MNCV was specifically altered and not the number of fibers being recruited during the electrical transmission because the amplitude of the compound motor action potential was similar between WT and αBC^{-/-} animals both before and after injury (FIG. 2 panels I, J). These results indicate that there was a delay in the recovery of normal electrophysiological properties of motor axons in null mice after crush injury.

αBC Positively Modulates Remyelination Following Sciatic Nerve Crush:

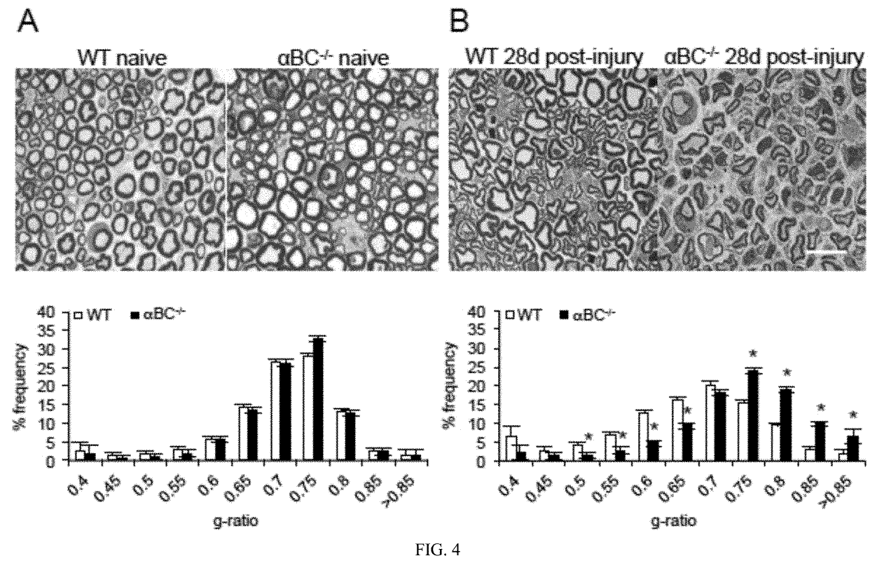

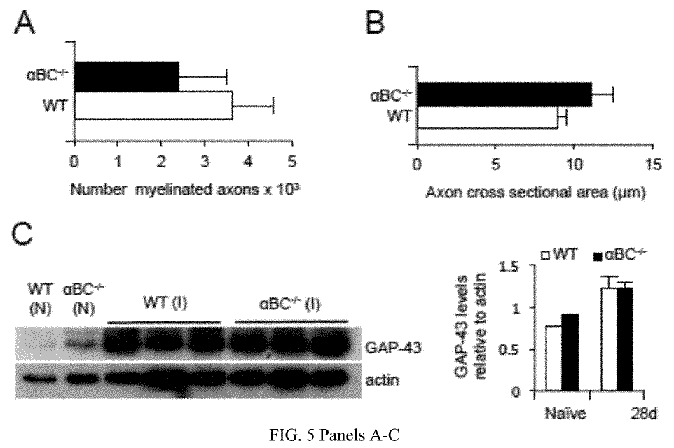

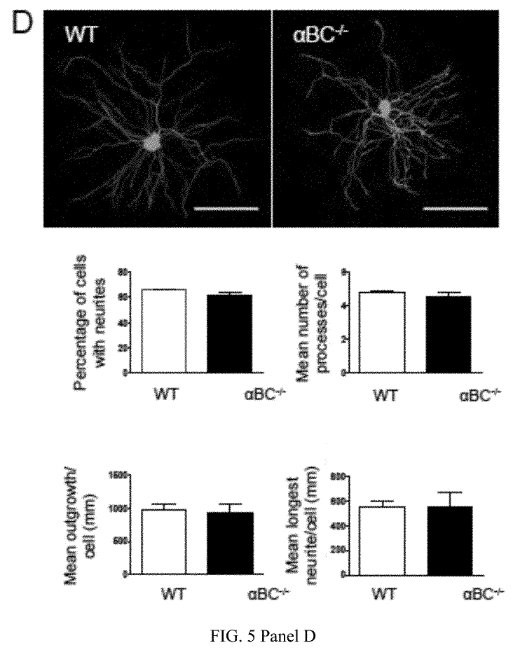

Since myelination and axonal integrity play a critical role in the electrophysiological properties of axons as well as motor and sensory behaviors, assessment was made to determine whether the defects seen in these parameters (FIGS. 2, 3) was because structural evidence of remyelination or axonal growth was different between WT and αBC^{-/-} animals at 28 d after injury, a time when these two events are robust in injured WT animals. For remyelination, g-ratios was quantified ranging from 0.4 to greater than 0.85, where 0.7 is the optimal myelin thickness for nerve conduction. Compared to injured WT nerves, it was found that the g-ratios in crushed αBC^{-/-} mice were skewed towards higher values (FIG. 4 panel B) which indicated that remyelination was impaired. Specifically, the frequency of low g-ratio profiles (axons with thick myelin sheaths) was significantly reduced in αBC^{-/-} animals whereas a marked increase in the frequency of high g-ratio values (indicative of axons with thin myelin sheaths) was greater in these animals as compared to their WT cohort (FIG. 4 panel B). The defect in remyelination exhibited by the αBC^{-/-} mice was not due to pre-existing differences in the naïve, uninjured mice but rather was related to the injury paradigm because the g-ratio profiles were equivalent in sciatic nerves from naïve, uninjured WT and null animals (FIG. 4 panel A). Furthermore, it appeared that myelin reformation was mainly targeted since the number of myelinated axons (FIG. 5 panel A) and the axon cross sectional area (FIG. 5 panel B) remained unchanged between injured WT and αBC^{-/-} mice. Additional evidence in support of this notion was that the levels of GAP-43, a growth associated protein, were equivalent in nerves from naïve and day 28 post-injured sciatic nerves from WT and null mice (FIG. 5 panel C). As well, the growth of neurites (outgrowth, number of processes, longest neurite) from cultured dorsal root ganglion (DRG) neurons was similar between the two genotypes (FIG. 5 panel D). Therefore, αBC^{-/-} mice displayed defects in remyelination following injury and possibly not growth of axons indicating that Schwann cells may be specifically impacted by the loss of the heat shock protein.

αBC Regulates Differentiation of Myelinating Schwann Cells:

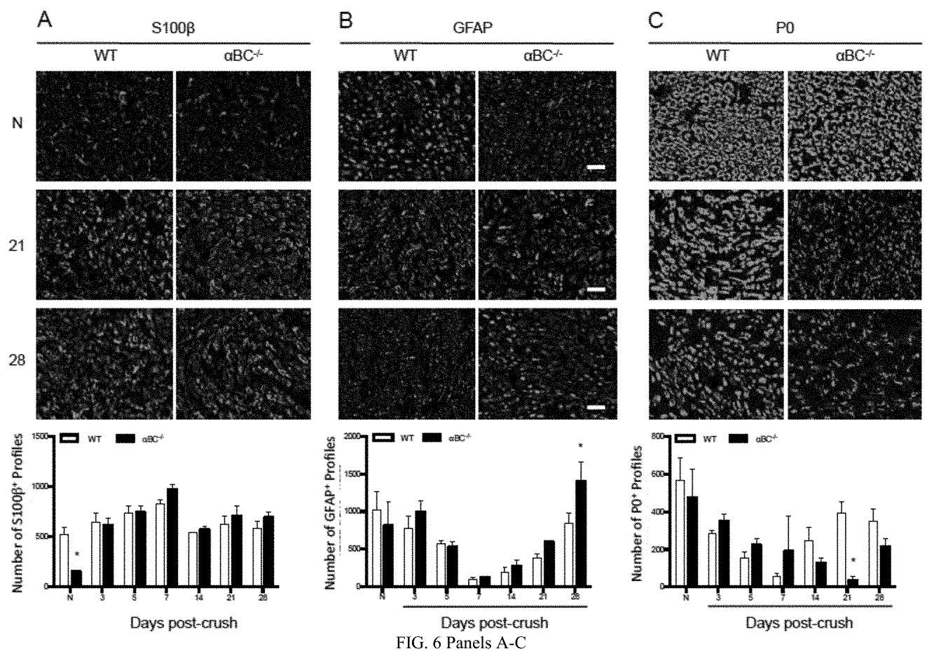

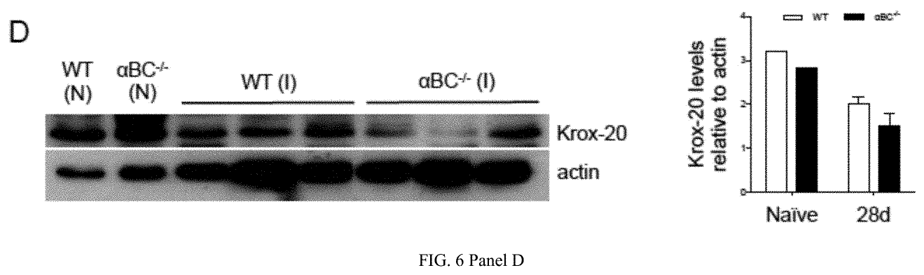

To identify the cellular mechanism(s) underlying the remyelination deficit in injured αBC deficient mice, the phenotype of Schwann cells was determined by quantifying the number of profiles in the distal nerve segment that were S100 (pan Schwann cell marker) positive (+), glial fibrillary acidic protein (GFAP)+(marker of de-differentiated or non-myelinating Schwann cells) and P0+ (myelinating Schwann cells). Although the number of S100+ profiles was equivalent between the WT and αBC^{-/-} groups from 3-28 d post-crush (FIG. 6 panel A), the proportion of GFAP+ and P0+ counts were markedly different between the two genotypes. Specifically, αBC^{-/-} animals had fewer numbers of P0+ profiles at 21 days post-injury and more GFAP+ cells at 28 d post-crush as compared to crushed WT nerves (FIG. 6 panels B, C). No difference in GFAP and P0 counts were seen at the earlier time points (3, 5, 7, 14 d) indicating that Schwann cell de-differentiation and proliferation were unaffected by αBC. Levels of Krox-20, a pro-myelinating transcription factor in Schwann cells, was measured, and it was found that damaged nerves from αBC deficient mice displayed a trend for reduced levels of Krox-20 relative to WT cohorts (FIG. 6 panel D). These data indicate that there may be a defect in the ability of αBC^{-/-} de-differentiated Schwann cells to switch back to a myelinating phenotype upon contact with regenerated axons.

NRG 1-ErbB2-AKT Axis is Modulated by αBC During Axonal Degeneration:

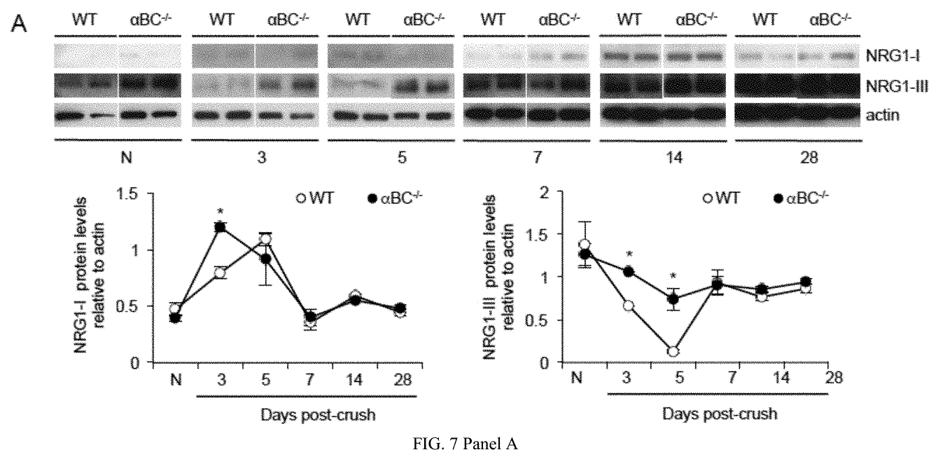

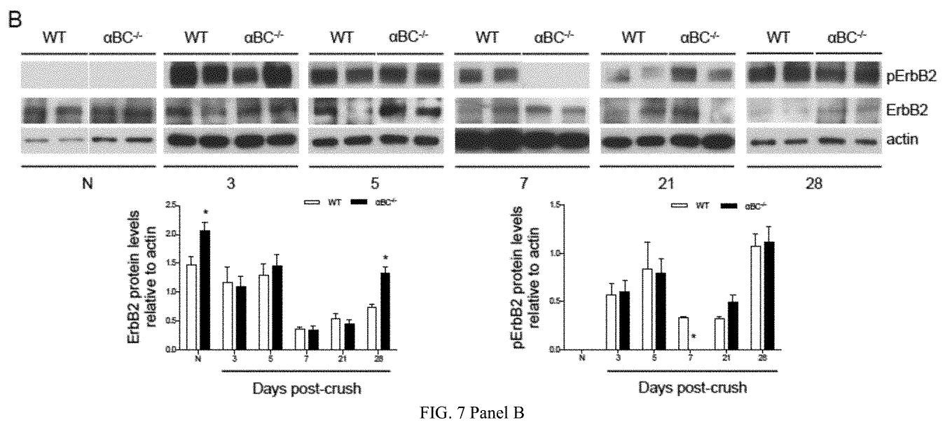

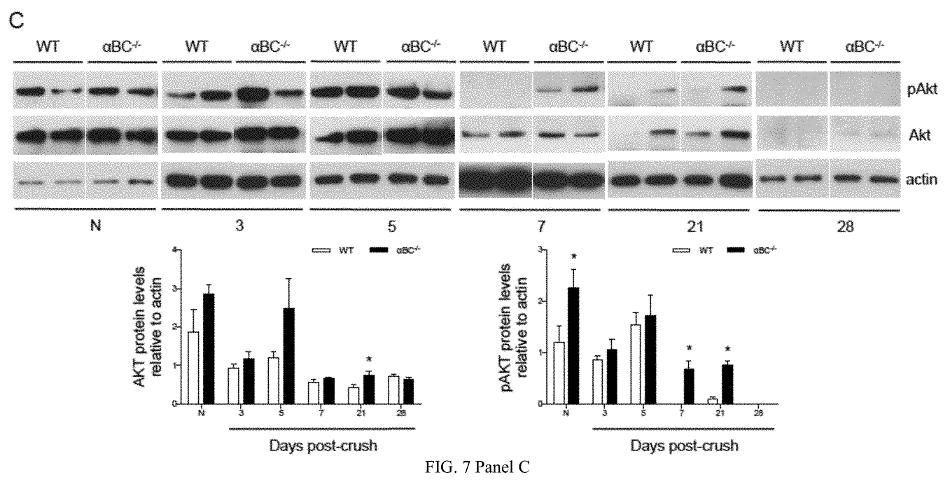

To assess for the molecular mechanisms driving αBC actions after PNS injury, as well as to ascertain whether early injury processes were impacted by the crystallin, the expression of neuregulin 1 Types I and III and its receptor ErbB2 was assessed. NRG 1-ErbB signaling is involved in many post-injury events including de- and remyelination, Schwann cell de- and re-differentiation, Schwann cell proliferation, re-myelination, regeneration and neuromuscular junction reinnervation. The levels of neuregulin 1 Type I increased after injury (within 3 d) before decreasing back to naïve levels by 7 days post-crush (FIG. 7 panel A). A similar temporal pattern was also seen in the αBC^{-/-} mice (FIG. 7 panel A) indicating that this Schwann cell-derived neuregulin is not involved in αBC-mediated injury processes. For neuregulin 1 Type III, its level decreased within 3 d after injury in WT animals and then rebounded to baseline status at 7 d post-crush (FIG. 7 panel B). This reduction however was minimal in the damaged null animals (FIG. 7 panel B) suggesting that this axon specific neuregulin was not responding appropriately to the injury. There is thus an axonal alteration in injured αBC^{-/-} mice in terms of NRG 1 Type III expression but this change did not impact number of myelinated axons, DRG process outgrowth or GAP-43 levels (FIG. 5). Regarding NRG1 receptors, assessment was made for the levels of phosphorylated and non-phosphorylated ErbB2. No significant difference was seen in the levels of non-phosphorylated ErbB2 between WT and null mice at 3, 5, 7 and 21 days after injury while higher levels were observed in the naïve and 28 d injured null animals (FIG. 7 panel B). With respect to phospho-ErbB2, no expression was visible in nerves from naïve WT and αBC^{-/-} mice but there was a robust increase in both WT and null animals within 3 d of crush injury. This enhancement in p-ErbB2 was maintained until day 28 in injured WT with a reduction evident midway at days 7 and 21 (FIG. 7 panel B). A similar pattern of p-ErbB2 expression was seen in the αBC^{-/-} animals but with an intriguing transient return to naïve levels 7 days after injury. Taken together, these findings indicate that αBC is involved in regulating NRG1 Type III-ErbB2 signaling in the early period after PNS injury.

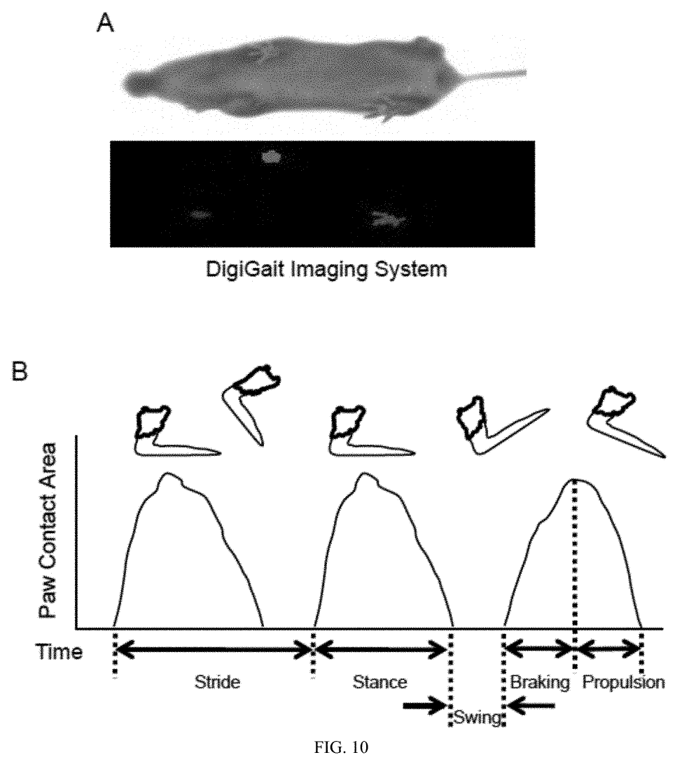

To delineate further the signal transduction pathway(s) that may be mediating the differences seen in NRG 1 Type III and phospho-ErbB2 in injured αBC^{-/-} mice, assessment was made for JNK, p38, ERK and AKT, pathways that are have been associated with PNS regeneration, Schwann cell properties, and αBC function. The levels of phospho JNK, p38 and ERK1/2 were significantly upregulated after injury in both WT and αBC^{-/-} relative to uninjured animals but there was no difference between the two genotypes post-crush (FIG. 10). With respect to AKT signaling, constitutive levels of AKT and p-AKT were present but not different between uninjured WT and null nerves (FIG. 7 panel C). In WT animals after damage, p-AKT expression remained similar to naïve levels until day 5 after which there was an almost complete loss of the transduction factor from days 7-28 post-crush. In αBC null animals, p-AKT levels at days 3 and 5 post-crush also remained similar to naïve levels. However, unlike the WT cohort, lower but detectable levels of p-AKT were still evident at days 7 and 21 post-crush and it was not until day 28 when the transduction factor was almost completely absent like in the WT animals. For both WT and αBC^{-/-} mice, AKT levels after injury remained similar to naïve animals at days 3 and 5 post-crush and reduced from days 7-28 but there was no overall difference between the two genotypes (FIG. 7 panel C). These findings indicate that αBC is involved in regulating NRG1 Type III-ErbB2 and pAKT signaling in the early period after PNS injury.

Exogenous Administration of αBC is Therapeutic after Sciatic Nerve Injury:

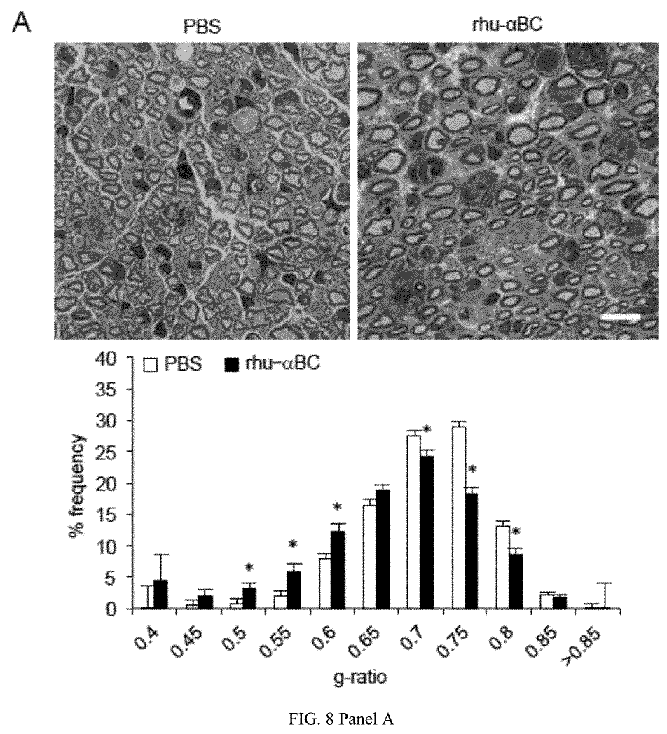

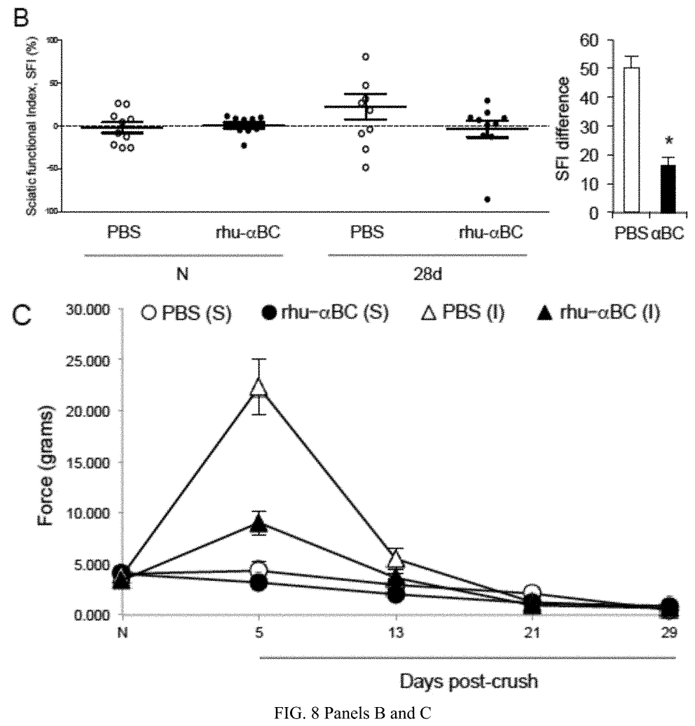

Evaluation was made on whether αBC can be therapeutic after peripheral nerve crush injury. Because the levels of endogenous αBC took several weeks to recover to baseline status after injury (FIG. 1 panel C), it was reasoned that exogenous application of the heat shock protein would enhance recovery processes. WT animals were injected every other day starting at day 1 after crush damage with either saline or recombinant human (rhu)-αBC. At 28 d post-damage, animals were subjected to behavioral testing and their nerves assessed for remyelination by g-ratio analysis. In terms of remyelination, mice injected with rhu-αBC displayed a skewing towards low g-ratios values which is indicative of thicker myelin sheaths. Specifically, the frequency of axons with small (thick myelin sheaths) and large (thinner myelin thickness) g-ratios was higher and lower respectively in the crystallin-treated group relative to controls (FIG. 8 panel A). With respect to functional recovery, injured WT mice treated with rhu-αBC displayed an SFI difference of 16.42±2.63 at 28 days post-crush whereas the PBS group was calculated at 50.09±4.06 (FIG. 8 panel B). These data indicated that walking ability had returned to almost pre-injury status at 28 d post-damage for WT animals treated with rhu-αBC as compared to the PBS cohort. The von Frey test was additionally implemented to test for sensory sensitivity. Here, both the PBS and αBC groups showed an augmentation in force at day 5 post-crush compared to the sham cohorts that then returned to baseline levels by day 13 post-damage (FIG. 8 panel C). However, the force needed to elicit a response was significantly lower in the αBC-treated group relative to the PBS-injected mice at day 5. Sensitivity early after sciatic nerve injury has been attributed to saphenous nerve sprouting in the medial and central areas of a mouse's paw and thus axon sprouting can be enhanced with exogenous application of rhu-αBC.

Discussion:

Heat shock proteins have been shown to be important for recovery after PNS and CNS nerve injury. Others have observed that Hsp27 promoted motor recovery after sciatic nerve damage while intravenous administration of recombinant human αBC was demonstrated to reduce lesion size and neuronal death and improve behavioral function following spinal cord injury. Because αBC is expressed in Schwann cells and axons, the present inventors conducted various experiments to determine whether the heat shock protein plays a role in the PNS. As shown herein, αBC can modulate post-injury processes following PNS damage. Based on the rapid reduction in expression of the crystallin within one day of sciatic nerve crush damage and its re-upregulation starting at 28 d post-crush (FIG. 1 panel C), it is believed that αBC was a negative regulator of the early events such as axon degradation, Schwann cell de-differentiation and Schwann cell proliferation and/or alternately as a positive modulator of regeneration and remyelination. The present inventors have demonstrate that αBC contributes to remyelination of peripheral axons since its absence attenuated myelin formation after crush injury. The present inventors have discovered that the remyelination defect is due to a reduced ability of de-differentiated Schwann cells to switch back to a myelinating phenotype following axon regeneration because of the reduced numbers of P0+ profiles and increased presence of GFAP+ cells in damaged nerves from αBC null mice relative to their WT counterparts. This may be driven by disruptions in NRG 1-Type III-ErbB2 signaling seen early after PNS damage in the null animals (FIG. 7). As one would expect with deficits in remyelination, defects in the electrophysiological properties of remyelinating axons (FIG. 3) were noted in injured αBC^{-/-} animals that likely contributed to the observed impairments in motor and sensory behaviors in the null mice (FIG. 2). Data shown herein also indicates that the dysfunctions in remyelination, behavior and electrophysiological properties in injured αBC null animals may not be related to defective axon regeneration since no difference in DRG neurite outgrowth, number of myelinated axons or levels of GAP-43 were seen between injured WT and αBC null mice. It is however possible that axon regrowth starts slowly after injury in the null animals and then accelerates to ‘catch up’ with WT mice at day 28 post-crush, or vice versa, starts fast and then slows down, both of which could impact remyelination. The present inventors have also shown that αBC has therapeutic use since injections of recombinant human αBC in injured WT animals enhanced remyelination and functional recovery after crush injury in WT animals (FIG. 8). Because of the reduced force needed to elicit a sensory response in injured animals treated with rhu-αBC at d5 post-crush (FIG. 8 panel C), it is believed that this is due to enhanced axonal sprouting by the saphenous nerve after sciatic nerve injury.

PNS Post-Injury Processes:

After PNS damage, an exquisitely orderly but overlapping sequence of processes occur in which alterations of early events (axon degeneration, Schwann cell de-differentiation, demyelination, Schwann cell proliferation and migration, immune cell infiltration) can impact later occurring functions such as axon regeneration, Schwann cell re-differentiation, remyelination and neuromuscular junction reinnervation. For example, in the Ola/WLD⁵ mouse in which axon degeneration is delayed by about two weeks, regeneration is impaired even though axon degeneration eventually occurs. This suggests that although regeneration would eventually proceed, albeit slowly, a rapid course of Wallerian degeneration is necessary if axons are to regenerate at optimal rates and to maximum extent. Others have found that in addition to delayed Wallerian degeneration, reduced PNS regeneration in injured Ola animals appears to be related to defects at the level of the neuronal cell body since neurite outgrowth is impaired if macrophages and their products are reduced or absent. This idea of early PNS injury events impacting later processes extends to other functions such as Schwann cell re-differentiation and remyelination. A number of seminal studies demonstrated that early upregulation of the MAP kinases, c-jun, ERK and p38 in Schwann cells after injury drove de-differentiation and proliferation of these glial cells and, that prolonging or eliminating their presence markedly altered myelin clearance, regeneration and remyelination. In addition, suppression or loss of NRGs or ErbBs which drives multiple aspects of Schwann cell and axon biology after injury such as de- and remyelination, Schwann cell de- and re-differentiation, regeneration and neuromuscular junction reinnervation, disrupt regeneration and remyelination.

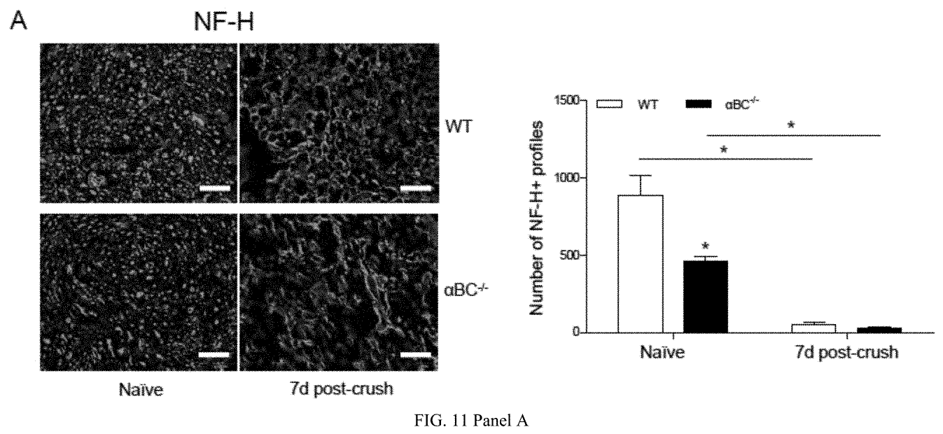

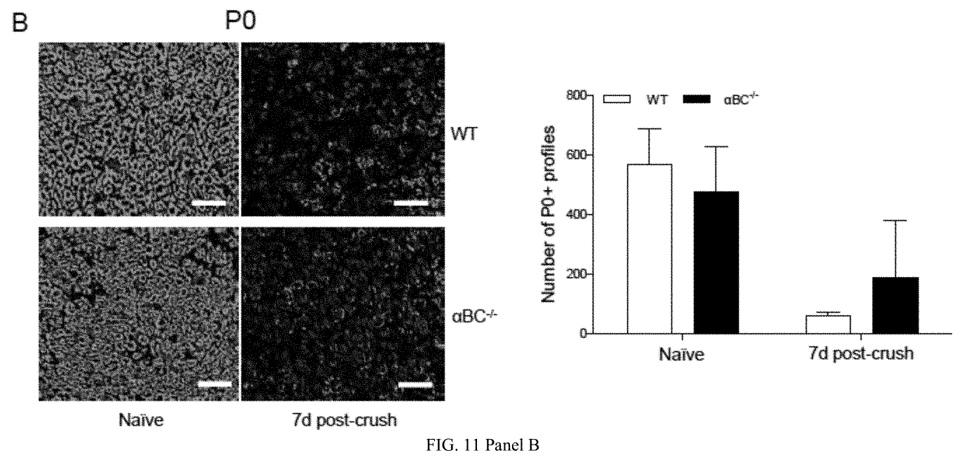

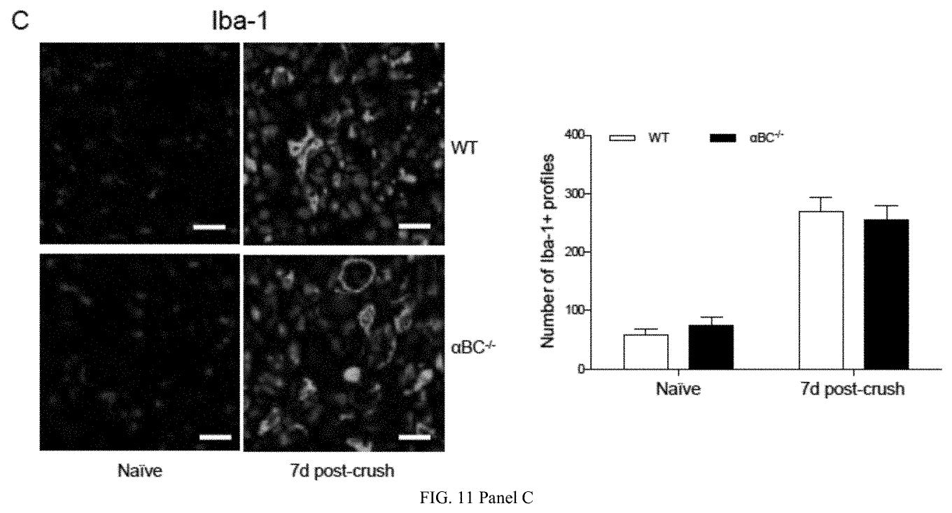

The present disclosure shows that the defect in remyelination (FIG. 4) and possible inability of de-differentiated Schwann cells to re-differentiate (FIG. 5) in injured null mice, is related to alterations in early injury events in the αBC^{-/-} mice. Evidence in support of this idea is that expression of NRG 1-III which normally declines after PNS injury remains elevated in the knockout animals after injury (FIG. 6 panel A). It is believed that the axons have not recognized that an injury has occurred and as a consequence respond inappropriately by maintaining constitutive NRG 1-III expression—as FIG. 11 shows, Wallerian and Wallerian-like processes such as neurofilament degeneration, myelin clearance and macrophage infiltration are occurring at equivalent levels in both injured WT and null mice. The unchanged NRG1-III levels in the αBC^{-/-} animals likely impacts later processes since a reduction in NRG 1-III initiates events such as Schwann cell de-differentiation and demyelination. Further, the transient near absence of ErbB2 expression at d7 post-crush in the null mice could also impact later remyelination. An increase in expression of ErbB2 was observed after sciatic nerve crush in WT animals but there was an interesting biphasic pattern in the WT animals where levels dipped at d7 post-crush before rebounding at day 28. The dual temporal responses can indicate a switch in functions for ErbB2. That is, the first increase could be related to early Schwann cell functions such as proliferation and the second to later events like remyelination. Others have also reported a biphasic response for ErbB2 where a transient increase in the first hour of peripheral nerve damage was associated with demyelination while a later increase around day 3 was associated with remyelination. In αBC^{-/-} mice, a similar biphasic response was evident but the transient loss of p-ErbB2 at d7, even though NRG 1-III levels had returned to baseline, could deviate the course of Wallerian degeneration and thus negatively impact later events that p-ErbB2 is involved in such as remyelination and re-differentiation. Some reported evidence in support of this idea is that the density of ErbBs appears to modulates NRG 1 activity and its absence can render Schwann cells insensitive to axonal NRG 1. Along the same lines, axonal NRG 1-IIII appears to act in a concentration dependent manner whereby Schwann cells display distinct responses, promyelination or myelin inhibition, depending on the levels of the NRG. Thus, changes in either NRG 1-III or ErbB2 would disrupt the communication between injured axons and Schwann cells and the many downstream processes they regulate such as remyelination. With respect to other properties of Schwann cells, the present data indicates that Schwann cell de-differentiation and proliferation are not impacted by αBC because the number of P0+ and GFAP+ profiles are equivalent between WT and null animals at all time points. There thus appears to be selectivity in the function of the heat shock protein after PNS damage.

Signal Transduction Signaling During Axonal Degeneration;

In an effort to identify the molecular mechanism(s) underlying the axonal degeneration changes in αBC null animals, assessment was made for MAP kinase and AKT signaling. The many reported functions of αBC such as cell survival, immunosuppression and chaperoning involve the JNK, p38 and ERK pathways. These signaling factors also participate in various aspects of Schwann cell function following peripheral nerve damage including Schwann cell de-differentiation and proliferation, regeneration, Schwann cell differentiation and remyelination. The present inventors found that MAP kinases were not altered before and after injury in the null mice as compared to WT counterparts indicating that these signal transduction factors do not universally mediate all functions of the heat shock protein. Surprisingly and unexpectedly, the present inventors discovered that the AKT pathway was associated with αBC function following peripheral nerve damage. The PI3K-AKT pathway has been implicated in PNS remyelination whereby increased levels or deficiency promoted or inhibited both PNS and CNS myelination. However, recent work by others noted that the PI3K pathway, which can act via AKT, has differing effects on Schwann cell myelination depending on its temporal expression. Early presence was associated with myelination via AKT/mTOR but later expression via laminin activation negatively affected myelination. In the present study, a significant increase in AKT after injury was not observed. Rather, an almost complete absence was clearly evident from d7 post-crush in WT animals while expression of the signal transduction factor was prolonged until much later at d28 in the null animals.

In addition to remyelination, it is also possible that αBC may be involved in triggering the degeneration process after PNS injury. Other have demonstrated that degradation of p-AKT levels was required for normal axon degeneration to occur after PNS injury. Breakdown of p-AKT releases inactivation of GSK3β which allows for phosphorylation of CRMP2 that is needed for microtubule reorganization (50). Thus, like WLD⁵ mice, the pronounced delay in reduction of p-AKT until day 28 after crush damage in αBC^{-/-} animals may negatively impact later post-injury processes like Schwann cell re-differentiation and remyelination.

As disclosed herein, αBC regulates specific events following damage to the PNS in the PNS. The present inventors have shown that Schwann cell re-differentiation and remyelination are regulated by αBC after peripheral nerve injury and, that these processes can be impacted by the heat shock protein also modulating events in the early phase of axonal degeneration such as NRG 1-III-ErbB2 signaling and possibly degeneration.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for treating a subject suffering from a peripheral nerve damage or injury, said method comprising administering to a subject in need of such a treatment a therapeutically effective amount of a molecule that increases remyelination of injured or damaged peripheral nerve cells.

2. The method of claim 1, wherein said molecule comprises alphaB-crystallin.

3. The method of claim 1, wherein said subject is treated with said molecule within one day of said peripheral nerve injury.

4. The method of claim 1, wherein said subject is treated with said molecule for at least 14 days after said peripheral nerve injury.

5. The method of claim 1, wherein said treatment results in at least 60% improvement in remyelination of injured or damaged peripheral nerve cells compared to the absence of said treatment.

6. The method of claim 1, wherein said peripheral nerve injury or damage comprises injury or damage to a sacral plexus nerve; a lumbar plexus nerve; a cranial nerve; a cervical plexus nerve; a brachial plexus nerve; a sympathetic nerve; a parasympathetic nerve; or a combination thereof.

7. The method of claim 6, wherein injury or damage to said sacral plexus nerve comprises injury or damage to sciatic nerve, sural nerve, tibial nerve, common peroneal nerve, deep peroneal nerve, superficial peroneal nerve, or a combination thereof.

8. The method of claim 6, wherein injury or damage to said lumbar plexus nerves comprises injury or damage to iliohypogastric nerve, ilioinguinal nerve, genitofemoral nerve, lateral cutaneous nerve, obturator nerve, femoral nerve, or a combination thereof.

9. The method of claim 6, wherein injury or damage to said cranial nerve comprise olfactory nerve, optic nerve, oculomotor nerve, trochlear nerve, abducens nerve, trigeminal nerve, facial nerve, vestibulocochlear nerve, glossopharyngeal nerve, vagus nerve, hypoglossal nerve, accessory nerve, or a combination thereof.

10. The method of claim 6, wherein injury or damage to said cervical plexus nerve comprises injury or damage to suboccipital nerve, greater occipital nerve, lesser occipital nerve, greater auricular nerve, lesser auricular nerve, phrenic nerve, or a combination thereof.

11. The method of claim 6, wherein injury or damage to said brachial plexus nerve comprises injury or damage to musculocutaneous nerve, radial nerve, median nerve, axillary nerve, ulnar nerve, or a combination thereof.

12. The method of claim 6, wherein injury or damage to said sympathetic nerve or said parasympathetic nerve comprises injury or damage to distal branches thereof.

13. The method of claim 1, wherein said method improves sensory activity of at least 80%.

14. The method of claim 1, wherein said method improves motor activity of at least 80%.

15. A method for treating a subject suffering from injured or damaged peripheral nerve, said method comprising administering to a subject suffering from injured or damaged peripheral nerve a therapeutically effective amount of alphaB-crystallin.

16. The method of claim 15, wherein said subject is treated with alphaB-crystallin within one day of suffering from injury or damage to peripheral nerve cell.

17. The method of claim 15, wherein said subject is treated with alphaB-crystallin for at least 14 days after suffering from injury or damage to peripheral nerve cell.

18. A method for treating a subject suffering from a peripheral nerve damage or injury, said method comprising treating said subject with a composition or a process to increase the expression level or availability of alphaB-crystallin.

19. The method of claim 18, wherein said composition comprises a therapeutically effective amount of a molecule that increases remyelination of injured or damaged peripheral nerves.

20. The method of claim 18, wherein said process to increase the expression level or availability of alphaB-crystallin comprises heat treatment, oxidative stress, osmotic dysregulation, or blocking a pathway known to inhibit alphaB-crystallin expression.

21. The method of claim 18, wherein said composition or process for increasing alphaB-crystallin expression or activity comprises heat, arsenite, phorbol 12-myristate 13-acetate, okadaic acid, H₂O₂, anisomycin, a high concentration of NaCl or sorbitol, or a combination thereof.
